(19) United States
(12) Plant Patent Application Publication
(10) Pub. No.: US 2002/0152523 P1
(43) Pub. Date: Oct. 17, 2002

(54) Strawberry plant named 'C216'

(76) Inventors: Kirk D. Larson, Irvine, CA (US); Douglas V. Shaw, Davis, CA (US)

Correspondence Address: BURNS DOANE SWECKER & MATHIS L L P, POST OFFICE BOX 1404, ALEXANDRIA, VA, 22313-1404, US

(21) Appl. No.: 09/776,754

(22) Filed: Feb. 6, 2001

(51) Int. Cl. A01H 5/00

(52) U.S. Cl. PLT/208

(57) ABSTRACT

A new and distinct short-day strawberry cultivar is provided. Attractive pointed symmetrical-conic bright light red fruit of good quality typically is formed in a good yield. The fruit flavor is good and the fraction of non-marketable fruit tends to be low. The growth habit is large, erect, and open. Flat to very concave leaflets having semi-pointed to pointed serrations are formed.

### FIELD OF THE INVENTION

A new and distinctive short-day type strawberry cultivar designated ‘C216’ is provided that resulted from a cross performed in 1996 between advanced selections Cal 93.170-606 (non-patented in the United States) and Cal 92.35-601 (non-patented in the United States). The cultivar is botanically identified as Fragaria×ananassa Duch. The parentage of the new cultivar can be summarized as follows:

Cal 93.170-606×Cal 92.35-601.

‘C216’ was first fruited at the University of California, South Coast Research and Extension Center near Irvine, Calif., U.S.A., in 1997, where it was selected, was originally designated Cal 96.42-601, and was propagated asexually by runners. The characteristics of the new cultivar have been found to be fully transmissible by such asexual propagation. Following selection and during testing the plant was designated ‘C216’. Asexual propagules from this original source have been tested at the Watsonville Strawberry Research Facility, the South Coast Research and Extension Center of the University of California, and to a limited extent in grower test fields starting in 1998.

It was found that the new cultivar of the present invention exhibits the following combination of characteristics:

(a) exhibits a large, erect and open growth habit,

(b) typically forms attractive pointed symmetrical-conic bright light red fruit of good quality and in a good yield, and

(c) forms flat to very concave leaflets having semi-pointed to pointed serrations.

### BRIEF DESCRIPTION OF THE PHOTOGRAPHS

The depicted plants and plant parts were grown at California, U.S.A.

FIG. 1—shows rows of typical fruiting plants during early to mid-March, 2000, at Irvine;

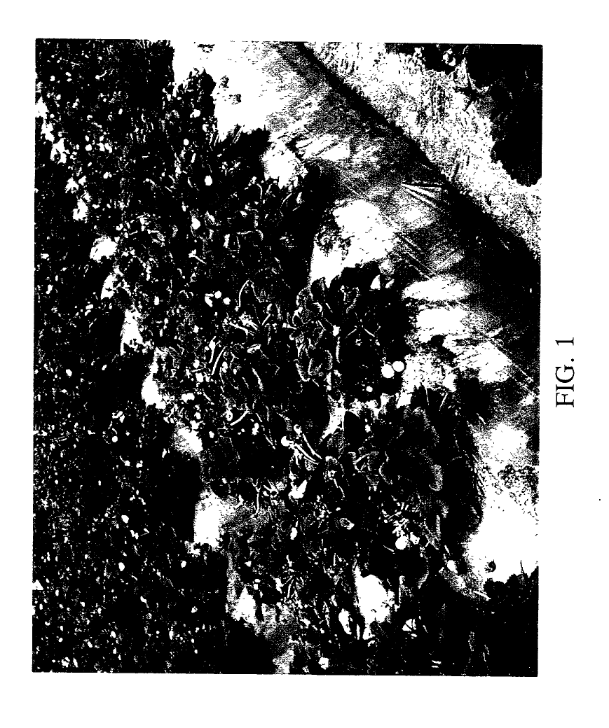

FIG. 2—shows a representative individual flowering and fruiting plant during early March, 2000, at Irvine wherein the erect and open growth habit is depicted;

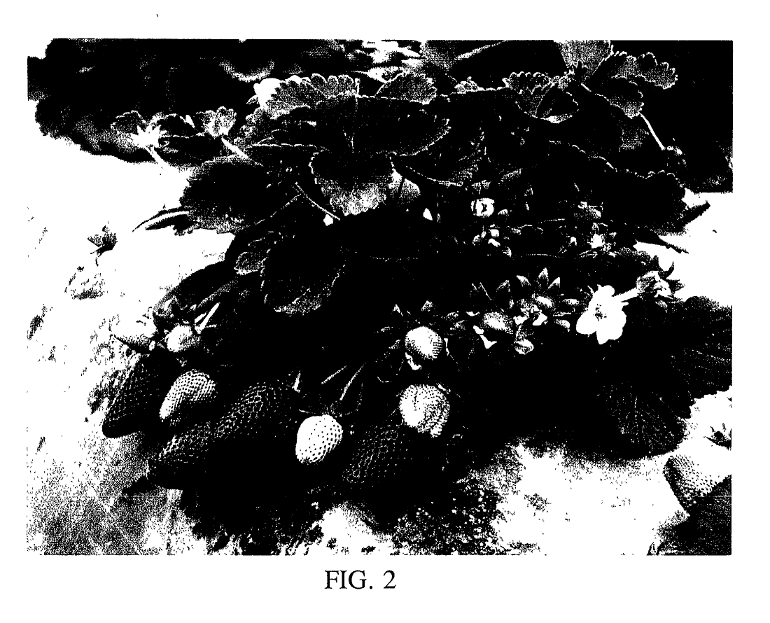

FIG. 3—shows a closer view of representative immature and mature fruit present on the plants;

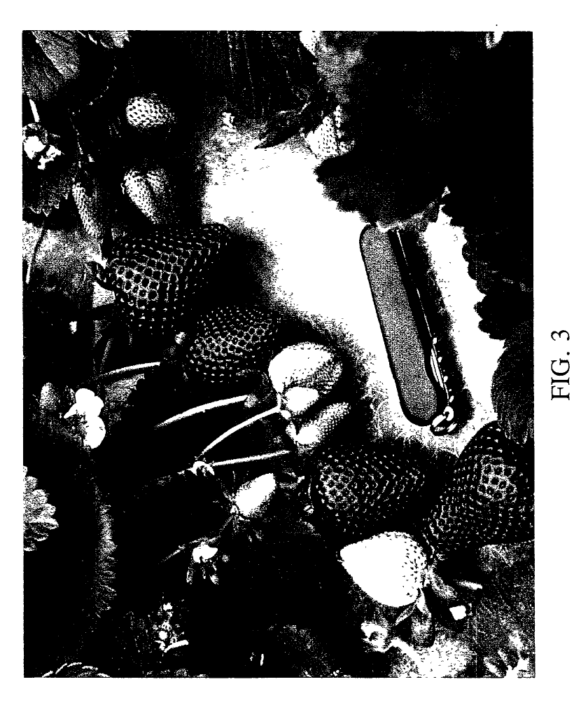

FIG. 4—shows a half-crate of representative fruit during mid-May, 2000, grown at Watsonville;

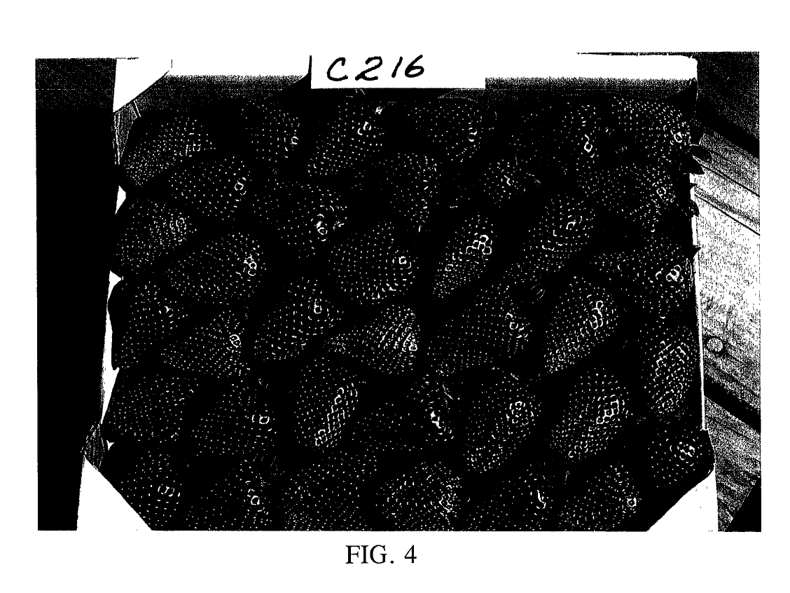

FIG. 5—shows a typical mature leaf during late spring.

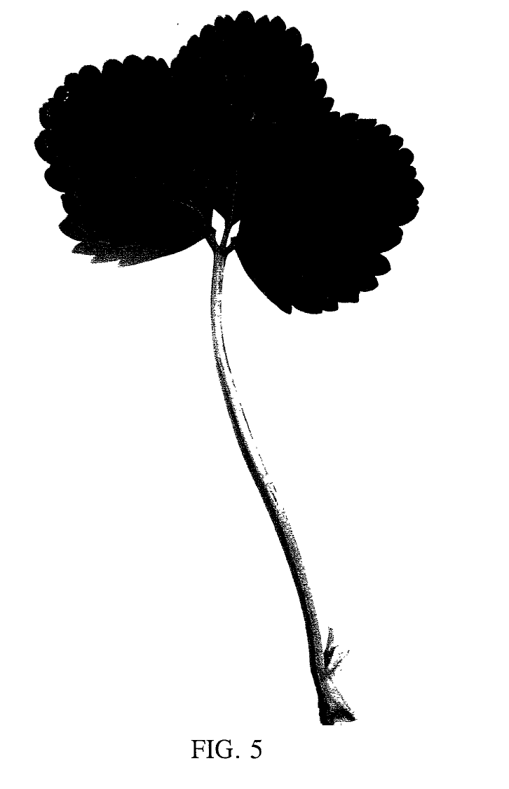

### DETAILED DESCRIPTION

‘C216’ as other short-day strawberry cultivars produces fruit over an extended period when treated appropriately in arid, subtropical climates. The production pattern for ‘C216’ is similar to that of the ‘Camarosa’ cultivar (U.S. Plant Pat. No. 8,708). It initiates fruiting at approximately the same time as the ‘Camarosa’ cultivar and produces greater quantities of early-season fruit with most cultural treatments. ‘C216’ will be of special interest for winter plantings where the ‘Camarosa’ cultivar has been successful, and in summer plantings where the ‘Pajaro’ cultivar (U.S. Plant Pat. No. 4,538) and the ‘Chandler’ cultivar (U.S. Plant Pat. No. 5,262) have been successful.

### Plants and Foliage

Fruiting plants of ‘C216’ are large and vigorous, similar to those of the ‘Camarosa’ cultivar, but more open than plants of the ‘Camarosa’ cultivar. ‘C216’ plants are larger and somewhat less erect than plants of the ‘Gaviota’ cultivar (U.S. Plant Pat. No. 10,461). Comparative statistics for foliar characters near mid-season are given for the three cultivars in Table 1 that follows. Individual leaflets for ‘C216’ are similar to those of the ‘Gaviota’ cultivar, and are somewhat more rounded than the leaflets of the ‘Camarosa’ cultivar. The leaves (including petioles) are longer and more narrow than those of the ‘Gaviota’ cultivar. The leaves are similar in size to those of the ‘Camarosa’ cultivar. The petioles are similar in thickness to those of the ‘Camarosa’ cultivar. ‘C216’ has a more concave leaf form than the ‘Gaviota’ and ‘Camarosa’ cultivars, and has more serrations and lighter leaf pubescence than the comparison cultivars.

TABLE 1

| | Cultivar | | |
|---|---|---|---|
| Foliar Character | ‘Camarosa’ | ‘Gaviota’ | ‘C216’ |
| Mid-Tier Leaflet | | | |
| Length (mm) | | | |
| mean | 91 | 95 | 93 |
| range | 80 to 100 | 83 to 109 | 75 to 115 |
| Width (mm) | | | |
| mean | 83 | 95 | 92 |
| range | 68 to 104 | 71 to 105 | 82 to 108 |
| Mid-Tier Leaf | | | |
| Length (mm) | | | |
| mean | 313 | 254 | 296 |
| range | 256 to 426 | 201 to 314 | 244 to 346 |
| Width (mm) | | | |
| mean | 161 | 182 | 174 |
| range | 130 to 183 | 146 to 205 | 134 to 205 |
| Number of | 3 | 3 | 3 |
| Leaflets/Leaf | | | |
| Leaf Convexity | flat-convex, | most are | flat to |
| | most are | concave, | very concave |
| | slightly | some flat | |
| | concave | | |
| Serrations | | | |
| number | few to | few to | moderate |
| | moderate | moderate | pointed |
| shape | rounded, some | rounded to | to semi-pointed |
| | semi-pointed | semi-pointed | |
| Leaf Pubescence | light-moderate | light-moderate | light |
| Petiole Pubescence | | | |
| density | moderate to | heavy | moderate |
| | heavy | | |
| direction | perpendicular | perpendicular | perpendicular |
| | to acropetal | | to slightly |
| | | | acropetal |

The adaxial (upper) surfaces of leaves of ‘C216’ are lighter in color than those of the ‘Gaviota’ cultivar and similar to those of the ‘Camarosa’ cultivar although they occasionally are more yellow. See Table 2 that follows.

TABLE 2

| Color Character | Cultivar | | |
|---|---|---|---|
| Leaf Color (CIELAB)* | ‘Camarosa’ | ‘Gaviota’ | ‘C216’ |
| Adaxial | | | |
| L* | | | |
| mean | 32.3 | 29.0 | 32.9 |
| range | 30.8 to 34.6 | 27.4 to 30.0 | 31.6 to 34.8 |
| a* | | | |
| mean | −7.0 | −7.0 | −7.9 |
| range | −4.8 to −8.2 | −6.1 to −7.7 | −7.0 to −9.4 |
| b* | | | |
| mean | 14.0 | 11.5 | 15.8 |
| range | 9.4 to 18.4 | 9.7 to 13.7 | 13.8 to 18.3 |
| Munsell | 5GY 4/3 | 5GY 4/3 | 10Y 4/3 |
| Adaxial | | | |
| L* | | | |
| mean | 46.1 | 47.5 | 48.1 |
| range | 43.9 to 49.2 | 46.4 to 48.7 | 46.1 to 49.7 |
| a* | | | |
| mean | −7.6 | −7.6 | −7.5 |
| range | −6.8 to −8.0 | −6.7 to −8.5 | −6.7 to −8.0 |
| b* | | | |
| mean | 21.8 | 20.4 | 19.0 |
| range | 20.1 to 25.0 | 17.2 to 24.5 | 17.5 to 23.3 |
| Munsell | 2.5GY 5/5 | 2.5GY 5/5 | 2.5GY 4/3 |

*CIELAB is the abbreviation of the international color system known as “Commission Internationale De L' Eclairage” 1978. For recommendations concerning uniform color spaces, color difference equations, and psychometric color terms see Supplement No. 2 of CIE Publication No. 15, Paris.

### Isozymes in Leaf Extracts

‘C216’ has been classified for three isozyme systems using Starch Gel Electrophoresis: Phosophoglucoisomerase (PGI), Leucine Aminopeptidase (LAP), and Phosphoglucomutase (PGM). It is distinguishable from the ‘Gaviota’ and ‘Camarosa’ cultivars using this methodology. See Table 3 that follows.

TABLE 3

| | Cultivar | | |
|---|---|---|---|
| Locus | ‘Camarosa’ | ‘Gaviota’ | ‘C216’ |
| PGI | A2 | A2 | A1 |
| LAP | B3 | B3 | B1 |
| PGM | C1 | C1 | C2 |

For electrophoretic procedures see: J. Amer. Soc. Hort. Sci. 106:684 to 687.

### Disease and Pest Reactions

‘C216’ is moderately susceptible to common leaf spot (Ramularia tulasnei) and Verticillium wilt (Verticillium dahliae). It is quite resistant to Phytophthora crown rot (Phytophthora cactorum), and relatively resistant to powdery mildew (Sphaerotheca macularis). When treated properly, it has tolerance to two-spotted spidermites (Tetranychus urticae) equal or greater to that of the ‘Gaviota’ and ‘Camarosa’ cultivars. ‘C216’ is tolerant to strawberry viruses encountered in California.

### Flowering, Fruiting. Fruit and Production Characteristics

Comparative statistics for flower and fruit characters near mid-season are given for ‘Camarosa’, ‘Gaviota’ and ‘C216’ in Table 4 that follows. The primary flowers for ‘C216’ are similar in size to those of the ‘Gaviota’ and ‘Camarosa’ cultivars although somewhat smaller in late spring. The sepals are somewhat smaller than those of the ‘Gaviota’ or ‘Camarosa’ cultivars. The calyx for ‘C216’ varies from even with the fruit shoulder to slightly necked, and each primary flower has 6 to 8 petals. The fruit shape for ‘C216’ is typically symmetrical conic, and is readily distinguishable from that of the ‘Camarosa’ cultivar that is shortened flat conic. ‘C216’ has a greater proportion of symmetrical fruit than the ‘Gaviota’ cultivar. Achenes vary from yellow to dark red, and are substantially even with the fruit surface or slightly indented.

TABLE 4

| | Cultivar | | |
|---|---|---|---|
| Character | ‘Camarosa’ | ‘Gaviota’ | ‘C216’ |
| Number of Petals | | | |
| mean | 6.3 | 5.9 | 6.7 |
| range | 5 to 8 | 5 to 8 | 6 to 8 |
| Flower Position | mostly even | exposed | even to |
| (relative to foliage) | some exposed | | exposed |
| Calyx Diam. (mm) | | | |
| mean | 48.1 | 41.7 | 35.1 |
| range | 32 to 58 | 30 to 58 | 31 to 45 |
| Corolla Diam. (mm) | | | |
| mean | 35.6 | 37.9 | 33.4 |
| range | 30 to 47 | 32 to 48 | 31 to 37 |
| Fruit Shape | | | |
| length/width | | | |
| ratio | 1.13 | 1.22 | 1.19 |
| range | 0.61 to 1.51 | 1.02 to 1.45 | 1.10 to 1.31 |
| subjective | Short flat | mostly rounded | Symmetrical |
| | conic | conic, some | conic |
| | | flat conic | |
| Calyx Position | even to | even to | even to |
| | slightly | slightly | slightly |
| | necked | indented | necked |
| Seed Position | even to | even | even to |
| | slightly | | extruded |
| | indented | | |

External and internal fruit color for ‘C216’ is lighter than that of the ‘Camarosa’ and ‘Gaviota’ cultivars, and is substantially brighter in coloration. See Table 5 that follows where CIELAB fruit color information is presented.

TABLE 5

| | Cultivar | | |
|---|---|---|---|
| | ‘Camarosa’ | ‘Gaviota’ | ‘C216’ |
| External | | | |
| L* | | | |
| mean | 23.3 | 22.4 | 23.8 |
| range | 20.7 to 27.0 | 20.3 to 24.5 | 17.5 to 28.4 |
| a* | | | |
| mean | 26.6 | 28.2 | 33.4 |
| range | 21.6 to 29.5 | 25.3 to 31.4 | 27.9 to 38.0 |
| b* | | | |
| mean | 12.8 | 14.8 | 19.4 |
| range | 9.9 to 14.6 | 12.0 to 17.7 | 13.3 to 28.2 |
| Munsell | 2.5R 3/7 | 5R 3/7 | 5R 4/12 |
| Internal | | | |
| L* | | | |
| mean | 44.0 | 48.1 | 50.7 |
| range | 40.7 to 46.9 | 44.3 to 53.9 | 46.2 to 60.0 |
| a* | | | |
| mean | 41.4 | 37.2 | 33.2 |
| range | 35.0 to 45.1 | 26.1 to 41.3 | 21.8 to 36.8 |
| b* | | | |
| mean | 29.9 | 28.6 | 24.5 |
| range | 24.1 to 35.3 | 19.9 to 32.6 | 16.4 to 27.5 |
| Munsell | 5R 4/12 | 5R 5/13 | 5R 5/13 |

### Performance

‘C216’ has been tested under a variety of cultural regimes, and optimal performance is obtained when nursery treatments and nutritional programs similar to those used with the ‘Camarosa’ cultivar are employed. When following appropriate planting regimes, ‘C216’ has similar fruit size and produces greater individual-plant yields than the ‘Gaviota’ or ‘Camarosa’ cultivars. In general, ‘C216’ is well adapted to very early season planting. ‘C216’ is earlier in its production pattern than the ‘Gaviota’ cultivar and similar to that of the ‘Camarosa’ cultivar, but with substantially greater production of early-season fruit (with conventional winter planting in southern or central California). Commercial appearance ratings have been better than those of the ‘Camarosa’ cultivar and trials conducted in Santa Maria, Calif., U.S.A. in 1999 to 2000 have indicated that the fraction of non-marketable fruit that is less than one-half that produced by the ‘Camarosa’ cultivar. Fruit for ‘C216’ is slightly firmer than fruit of the ‘Gaviota’ cultivar, and slightly less firm than that of the ‘Camarosa’ cultivar. Subjectively, ‘C216’ has very good flavor. The fruit will be outstanding for both fresh market and processing, and will be useful for home garden purposes. See the comparative data presented in Table 6 that follows. There information is provided for plants that were evaluated during 1999 and 2000 at the South Coast Research and Extension Center of the University of California near Irvine, Calif., U.S.A. Such plants had been asexually reproduced by the use of runners at Macdoel, Calif., U.S.A., were harvested on October 1^{st} to 3^{rd}, and were transplanted approximately 2 to 4 days after harvest. The test planting consisted of 64 inch four-row beds, 21,780 plants/acre. Harvest was initiated in late December and continued through the last week of June.

TABLE 6

| | Early | | Appear- | Fruit | |
|---|---|---|---|---|---|
| | Yield* | Yield | ance | Size | |
| Cultivar | (g/plant) | (g/plant) | Score | (g/fruit) | Firmness |
| ‘Camarosa’ | 254 | 1,271 | 3.0 | 29.3 | 3.2 |
| ‘Gaviota’ | 179 | 764 | 2.8 | 29.5 | 2.4 |
| ‘C216’ | 277 | 1,511 | 3.3 | 31.1 | 3.5 |

*Marketable yield to March 10.
‘C216’ has not been observed under all possible environmental conditions to date. Accordingly, it is possible that the phenotype may vary somewhat with variations in the environment.

We claim:

1. A new and distinct short-day strawberry plant cultivar that exhibits the following combination of characteristics:
(a) exhibits a large, erect and open growth habit,
(b) typically forms attractive pointed symmetrical-conic bright light red fruit of good quality and good yield, and
(c) forms flat to very concave leaflets having semi-pointed to pointed serrations; substantially as illustrated and described.
